(19) United States
(12) Patent Application Publication
(10) Pub. No.: US 2015/0018647 A1
(43) Pub. Date: Jan. 15, 2015

(54) METHOD AND APPARATUS FOR MONITORING A SUBJECT FOR BLOOD OXYGEN SATURATION

(71) Applicant: Xerox Corporation, Norwalk, CT (US)

(72) Inventors: Barry P. MANDEL, Fairport, NY (US); Peter Johan NYSTROM, Webster, NY (US); Lalit Keshav MESTHA, Fairport, NY (US)

(73) Assignee: XEROX CORPORATION, Norwalk, CT (US)

(21) Appl. No.: 13/937,782

(22) Filed: Jul. 9, 2013

(51) Int. Cl. A61B 5/1455; A61B 5/00

(52) U.S. Cl. CPC A61B 5/14552; A61B 5/681; A61B 5/6829; A61B 5/7282; A61B 5/742; A61B 5/0022
USPC 600/323

(57) ABSTRACT

What is disclosed is a system and method for monitoring a subject of interest for functional blood oxygen saturation using an apparatus that can be comfortably worn by the subject around an area of exposed skin where a photoplethysmographic (PPG) signal can be registered. In one embodiment, the apparatus is a reflective or transmissive wrist-worn device with emitter/detector pairs fixed to an inner side of a band with at least two illuminators, each emitting source light at a different wavelength band. Each photodetector comprises sensors that are sensitive to a wavelength band of its respective illuminator. Each photodetector measures an intensity of sensed light emitted by a respective illuminator. The signal obtained by the sensors comprises a continuous PPG signal. The continuous PPG signal analyzed for functional blood oxygen saturation levels and communicated to a remote device. Various embodiments are disclosed.

### CROSS REFERENCE TO RELATED APPLICATIONS

This patent application is related to concurrently filed and commonly owned U.S. patent application Ser. No. 13/937,740, “Method And Apparatus For Monitoring A Subject For Atrial Fibrillation”, by Mestha et al. (Attorney Docket 20121584-US-NP), and U.S. patent application Ser. No. 13/______, “Method And Apparatus For Monitoring A Subject For Fractional Blood Oxygen Saturation”, by Mestha et al. (Attorney Docket 20121584Q1-US-NP), which are incorporated herein in their entirety by reference.

### TECHNICAL FIELD

The present invention is directed to an apparatus that can be worn circumferentially around an area of exposed skin of a subject being monitored for a functional blood oxygen saturation level.

### BACKGROUND

Hemoglobin, also spelled haemoglobin, (Hb, also written Hgb) is the iron-containing oxygen transport metalloprotein in red blood cells of most vertebrates and some invertebrates. Heme sites within a blood cell contain ferrous [Fe²⁺] ion which has an affinity to oxygen (binds lightly to oxygen). The mammalian Hb molecule has a complex quaternary structure with each Hb molecule having two alpha chain and two beta chain polypeptides. Each Hb chain contains a single molecule of heme (a heme unit). Each unit of heme holds an Fe⁺² ion (also written Fe²⁺) in such a way that the iron can interact with an oxygen molecule to form oxygenated hemoglobin or oxyhemoglobin (O₂Hb, also written HbO₂). If the hemoglobin is fully saturated then it can transport four oxygen molecules from the respiratory organs (lungs or gills) where the oxygen is received, to body tissues where the heme sites release the oxygen they are carrying. Oxygenated blood is bright red due to the iron being bound to oxygen. The oxygen is used by the cells to burn nutrients which, in turn, provide energy to power cellular metabolisms. The heme sites collect carbon dioxide (CO₂) and returns these gases back to the respiratory organs to be expelled into the surrounding environment via a process of exhalation or expiration. Deoxygenated blood has a bluish color. There is a need for a device that can provide abilities to continuously monitor patient's functional oxygen level seamlessly without impeding their mobility, causing discomfort or limiting the use of their hands using reflective or transmissive sensing arrangement. The device can be worn on the wrist, ankle, arm or leg. The information gathered from the device can be transmitted to a smart phone using wireless (e.g. Bluetooth or NFC) or wired technology, where it can be analyzed, displayed and further transmitted to a central monitoring station via the internet.

Accordingly, what is needed in this art is a method and apparatus for monitoring a subject of interest for functional blood oxygen saturation levels which can be comfortably worn by the subject circumferentially around an area of exposed skin.

### BRIEF SUMMARY

What is disclosed is a method and apparatus for monitoring a subject of interest for functional blood oxygen saturation. Methods are disclosed for determining a functional oxygenation saturation level using either a reflective or a transmissive sensing apparatus. Each embodiment comprises emitter/detector pairs fixed to an inner side of a band worn circumferentially around an area of exposed skin of a subject with at least two illuminators each emitting source light at a different wavelength band. In one embodiment where the present apparatus comprises a transmissive sensing device, each photodetector measures an intensity of light emitted from its respective paired illuminator which has passed through a chord of living tissue. In another embodiment where the present apparatus comprises a reflective device, each photodetector measures an intensity of light emitted from its respective paired illuminator which has reflected off a surface of the skin. In each configuration, a time-series signal is generated by the continuous sensing of light intensities. The time-series signal comprises a continuous PPG signal of the subject. In another embodiment, the time-series signal is processed to extract the continuous PPG signal. Both embodiments are disclosed herein. The continuous PPG signal is analyzed to determine functional blood oxygen saturation levels. Alert signals can be communicated to one or more remote devices such as, a smartphone, if the monitored levels fall outside a limit of acceptability pre-set for this subject.

Many features and advantages of the above-described apparatus will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 illustrates an anterior and dorsal view of a subject of interest to show various locations where the present apparatus is likely to be worn;

FIG. 2 illustrates the anterior and dorsal views of the subject of FIG. 1 showing that the present apparatus can also be worn circumferentially around the neck and circumferentially around an area of the subject's mid-section;

FIG. 3 shows an embodiment of the present apparatus being worn circumferentially around a finger of each of the subject's left and right hands;

FIG. 4 shows one embodiment of a transmissive device worn circumferentially around an area of exposed skin as shown in any of FIGS. 1-3;

FIG. 5 shows one embodiment of a reflective device worn circumferentially around the area of exposed skin of FIGS. 1-3;

FIG. 6 shows the angular relationships of the emitter/detector pair of the reflective device of FIG. 5;

FIG. 7 shows one embodiment of a control panel fixed to an outer side of the bands of each of the transmissive and reflective devices of FIGS. 4 and 5;

FIG. 8 illustrates Beer's Law in a tissue layer containing venous and arterial structures;

FIG. 9A plots extinction coefficients for RHb, HbO₂, COHb, and MetHb;

FIG. 9B plots extinction coefficients of RHb and HbO₂ in regions of red and infrared wavelengths;

FIG. 10 is a flow diagram of one embodiment of the present method for monitoring a subject of interest for functional blood oxygen saturation; and

FIG. 11 is a block diagram illustrating one example networked system for performing various aspects of the teachings hereof;

### DETAILED DESCRIPTION

What is disclosed is a system and method for monitoring a subject of interest for functional blood oxygen saturation using an apparatus which can be comfortably worn around an extremity. The present apparatus is disclosed in two embodiments, i.e., a reflective sensing device and a transmissive sensing device.

### Non-Limiting Definitions

A “subject of interest” refers to a subject having a cardiac function. Although the term “human”, “person”, or “patient” may be used throughout this disclosure, it should be appreciated that the subject may not be human. As such, use of the terms “human”, “person” or “patient” is not to be viewed as limiting the scope of the appended claims strictly to human beings.

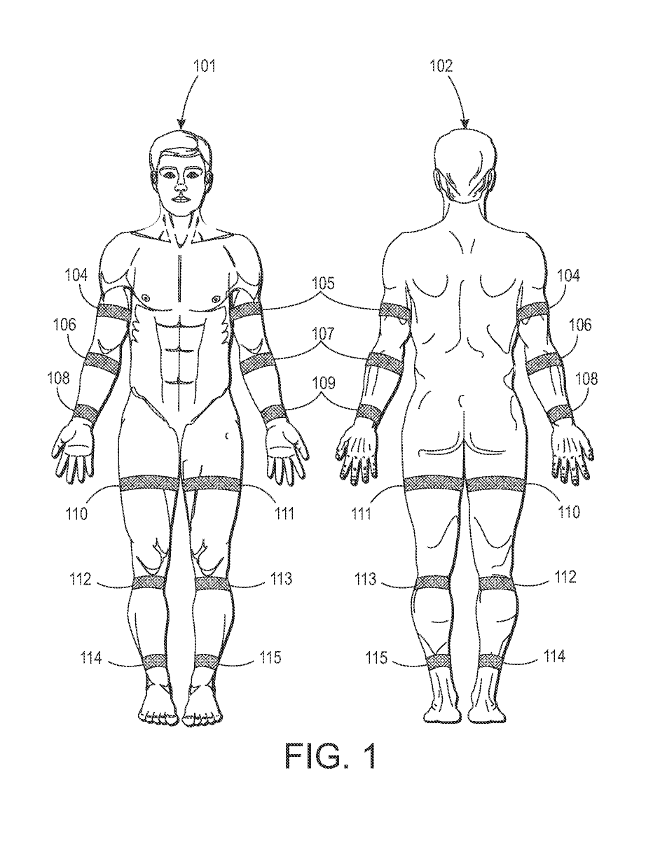

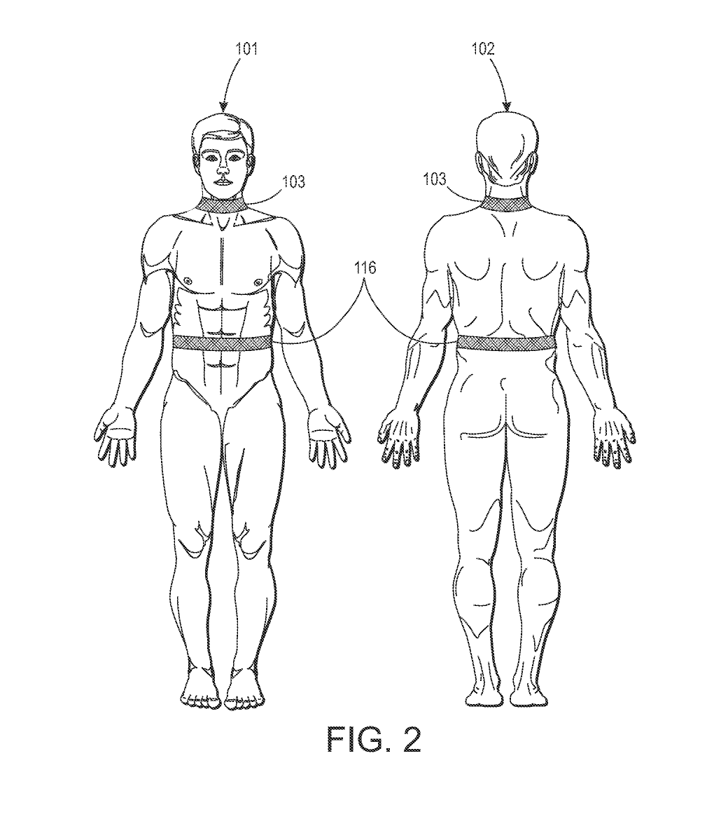

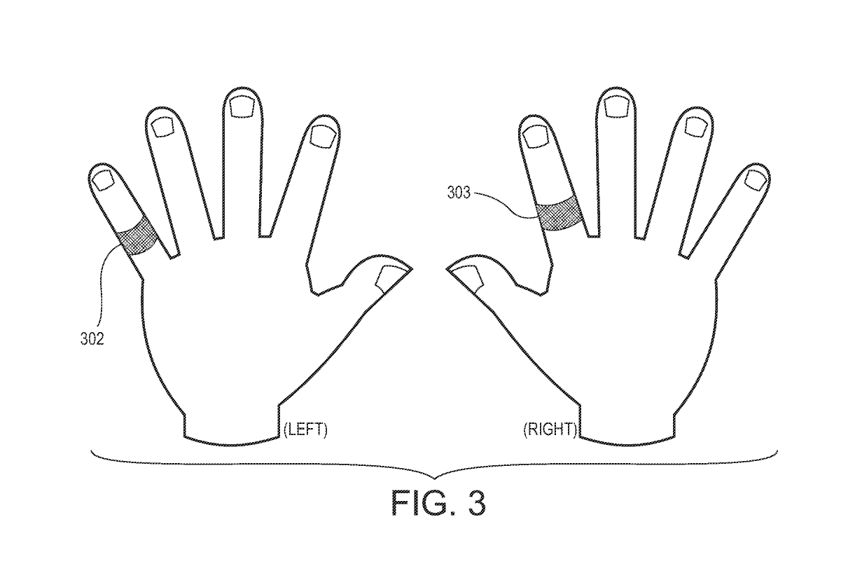

An “area of exposed skin” refers to a circumferential region of the subject where a photoplethysmographic (PPG) signal can be obtained by various embodiments of the apparatus disclosed herein. FIG. 1 illustrates an anterior view 101 of a subject of interest and a dorsal view 102. Various circumferential areas of exposed skin are shown where the present apparatus is likely to be worn. For example, the present apparatus can be worn circumferentially around the upper left or right arms at 104 and 105, respectively. Or, around the left or right forearms at 106 and 107; around the left or right wrists at 108 and 109; around the upper left and right thigh at 110 and 111; around the left and right calf at 112 and 113; or around the left and right ankle at 114 and 115. FIG. 2 illustrates the anterior and dorsal views of the subject of FIG. 1 showing that the present apparatus can also be worn circumferentially around the neck 103 and around an area of the mid-section 116 where PPG signals can be registered. The illustrations of FIGS. 1 and 2 should not be viewed as limiting the scope hereof to the areas shown, as other embodiments of the present apparatus can be worn circumferentially around the finger, toe, forehead, hand, and foot. FIG. 3 shows an embodiment of the present apparatus being worn circumferentially around a finger 302 of the subject's left hand or around a finger 303 of the subject's right hand.

“Photoplethysmography” is the study of signals containing relative blood volume changes in vessels which are close to the skin surface. Sensors of the present apparatus using sequentially captured pulsating signals provide a continuous time-series signal which, in one embodiment, is the subject's PPG signal. In other embodiments, the time-series signal is processed to extract the PPG signal. In this alternative embodiment, a sliding window is used to define consecutive time-sequential segments of the time-series signal. Each signal segment overlaps a previous segment by at least a 95%. Each of the consecutive time-series signal segments is detrended to remove low frequency variations and non-stationary components. The detrended signal segments are filtered such that frequencies of the subject's cardiac beat are retained. In one embodiment, the filter comprises a higher-order band-limited Finite Impulse Response (FIR) Filter which constrains band width to a desired range of the subject's heart. The filtered time-series signal segments are then upsampled to a pre-selected sampling frequency to increase a total number of data points in order to enhance an accuracy of peak-to-peak pulse point detection. In one embodiment, upsampling involves an interpolation technique using a cubic spline function and a pre-selected sampling frequency. The upsampled time-series signal segments are then smoothed using any of a variety of smoothing techniques. These processed signal segments are then stitched together to obtain a continuous PPG signal for the subject. Method for obtaining a continuous PPG signal are disclosed in: “Continuous Cardiac Pulse Rate Estimation From Multi-Channel Source Video Data”, U.S. patent application Ser. No. 13/528,307, by Kyal et al., “Continuous Cardiac Pulse Rate Estimation From Multi-Channel Source Video Data With Mid-Point Stitching”, U.S. patent application Ser. No. 13/871,728, by Kyal et al., and “Continuous Cardiac Signal Generation From A Video Of A Subject Being Monitored For Cardiac Function”, U.S. patent application Ser. No. 13/871,766, by Kyal et al., all of which are incorporated herein in their entirety by reference.

An “emitter” refers to an illuminator which emits source light at a desired wavelength band. An emitter may comprise one or more illuminators. Functional oxygen saturation determination requires at least two illuminators. In one embodiment, a wavelength band of a first illuminator is centered about 660 nm and a wavelength band of a second illuminator is centered about 940 nm.

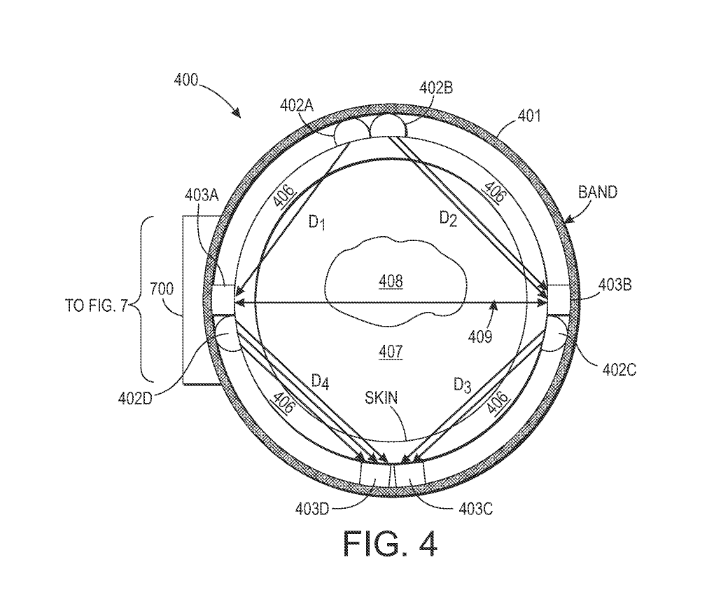

A “photodetector” or simply “detector” is a light sensing element comprising one or more sensors or sensing elements which are sensitive to a wavelength band of a respective illuminator system. Each photodetector continuously measures an intensity of received light emitted by its illuminators and outputs, in response thereto, a time-series signal. To improve signal to noise ratio in the time-series signal, in one embodiment, all the emitters at similar wavelength band are illuminated and photodetector outputs combined to produce a single time-series signal. The photodetectors are fixed to an inner side of the band with each emitter/detector pair being separated by a distance D, as discussed with respect to FIGS. 4-6.

A “transmissive device” is one embodiment of the present apparatus where the distance D separating each illuminator and paired detector defines a chord of living tissue through which the emitted source light passes. The distance D is less than 75% of a diametrical distance of the area around which the apparatus is worn. The respective paired photodetector measures an intensity of light passing through the chord of living tissue. FIG. 4 shows one embodiment of a transmissive device 400 worn circumferentially around an area of exposed skin. Band 401 has a plurality of emitter/detector pairs fixed to an inner side thereof. Emitters 402A-D are paired, respectively, to detectors 403A-D. Emitter 402A comprises a single illuminator which emits light at a desired wavelength band. Emitters 402B and 402C each comprise two illuminators, which may emit light at the same or different wavelength bands. Emitter 402D is shown comprising three illuminators which may all emit source light at a same or different wavelength bands. The band 401 may comprise any configuration of emitter/detector pairs. FIG. 4 is one example. Band 401 is worn circumferentially around an area the skin 406 covering a plurality of subcutaneous tissues (collectively at 407) which surround deeper tissues such as muscles, organs, bones, and the like (collectively at 408). Distances D₁, D₂, D₃ and D₄ define a chord of living tissue through which light emitted by illuminators 402A-D passes and which, in turn, is detected by paired photodetectors 403A-D. Although not illustrated exactly to scale, distance D₁, D₂, D₃, D₄ are less than 75% of a diametrical distance 409 of the area around which the apparatus is worn. Distances between respective emitter/detector pairs do not have to be equal. It should be appreciated that the subcutaneous tissues include a plurality of blood vessels and other tissue structures. Other embodiments of the transmissive device hereof comprise multiple emitter/detector pairs fixed to an inner side of the band.

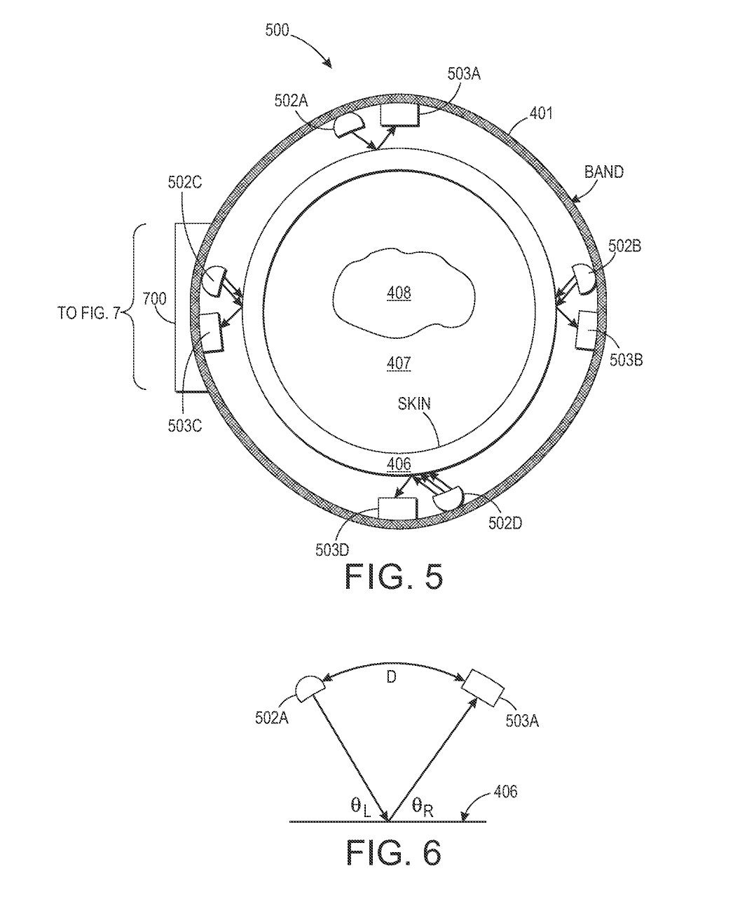

A “reflective device” is one embodiment of the present apparatus where each photodetector measures an intensity reflecting off a surface of skin 406. FIG. 5 shows one embodiment of a transmissive device 500 worn circumferentially around an area of exposed skin as shown in any of FIGS. 1-3. Band 401 has a plurality of emitter/detector pairs fixed to an inner side thereof. Emitters 502A-D are paired, respectively, to detectors 503A-D. Emitter 502A comprises a single illuminator which emits light at a desired wavelength band. Emitters 502B and 502C each comprise two illuminators, which may emit light at the same or different wavelength bands. Emitter 502D is shown comprising three illuminators which may all emit source light at a same or different wavelength bands. The band 401 may comprise any configuration of emitter/detector pairs. FIG. 5 is one example. Band 401 is worn circumferentially around an area the skin 406 covering a plurality of subcutaneous tissues (collectively at 407) which surround deeper tissues such as muscles, organs, bones, and the like (collectively at 408). It should be appreciated that the subcutaneous tissues include a plurality of blood vessels and other tissue structures. In FIG. 6, the source light emitted by each illuminators 502 impacts the surface of the skin 406 at angle θ_{L} and reflects off the skin surface at angle θ_{R}, where 0°<(θ_{L}, θ_{R})<90°. Distance D is a distance measured between each illuminator 502 and its paired photodetector 503.

### Example Control Panel

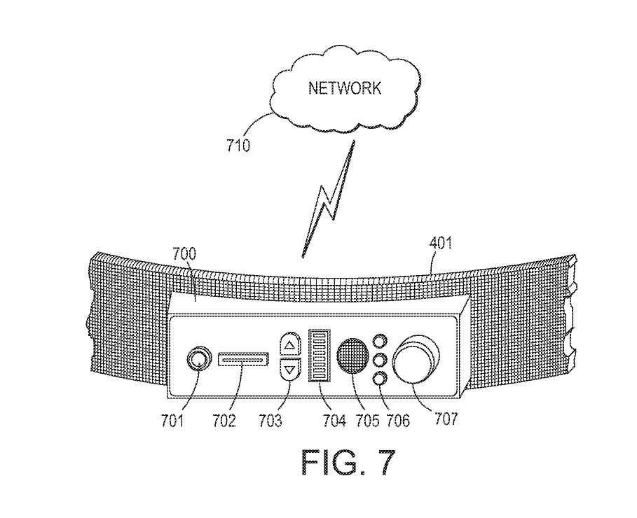

Reference is now being made to FIG. 7 which shows one embodiment of a control panel 700 fixed to an outer side of the bands of each of the transmissive and reflective devices of FIGS. 4 and 5. The control panel allows the user to effectuate various aspects of the functionality of the embodiments disclosed herein.

In FIG. 7, the control panel 700 has a female adaptor 701 for receiving male counterpart of a power supply, as are normally understood, to charge one or more batteries (not shown). In some embodiments, a separate power supply comprising a battery pack is kept in a pocket and a cord is connected to the control pattern via adaptor 701. The power supply may be a transformer plugged into a wall socket with a cord which provides continuous power to the present apparatus. Also shown is a slot 702 for insertion of a memory chip or MicroSD card as are typically found in cellular smartphone devices. Such a removable memory card records signals obtained from the photodetectors, and may contain device specific parameters which are used to set power levels, adjust intensity values, provide data, formulas, threshold values, patient information, and the like. Once inserted into the device, the present apparatus reads the data as needed. A microprocessor (CPU) or ASIC internal to the control panel would read the removable card including uploading executing machine readable program instructions contained thereon for performing any of the functionality described herein.

Directional buttons 703, are shown to enable a variety of functions including increasing/decreasing a volume being played through speaker 704. The up/down buttons may be configured to increase intensities of any of the emitters fixed to an inner side of band 401 or to adjust the sensitivities of any of the sensor elements of the photodetectors. Buttons 704 may be used to tune the present apparatus to standards set by the FDA or other regulatory agencies. USB port 704 enables the connection of a USB cord to the present apparatus. Such a connection can enable any of a variety of functions. For example, the USB device may be used to program a microprocessor or configure the present apparatus specifically to a particular patient and set threshold levels for blood oxygen saturation detection and monitoring.

Speaker 705 enables an audible feedback for the visually impaired. Such as an audible alert may be initiated in response to the detected blood oxygen saturation being outside a pre-defined limit of acceptability. The audible alert may be varied in volume, frequency, and intensity, as desired, using the up/down buttons 703. LEDs 706 enable any of a variety of visual feedback for the hearing impaired. Visual feedback may take the form of, for instance, a green LED being activated when the device is turned ON. A red LED may be activated in response to an alert condition. A blue LED can be activated when the a physiological even is not present. The LEDs can be activated in response to a communication occurring between the present apparatus and a remote device via a wireless communication protocol. The LEDs may be activated in combination. Button 707 turns the device ON/OFF. The device is capable of wirelessly communicating text, email, picture, graph, chart, and/or a pre-recorded message to a remote device such as, for example, a smartphone, a Wi-Fi router, an I-Pad, a Tablet-PC, a laptop, a computer, and the like. Such communication may utilize a Bluetooth protocol. The communication may utilize network 710 shown as an amorphous cloud.

It should be appreciated that the embodiments described are illustrative for explanatory purposes and are not to be viewed as limiting the scope of the appended claims solely to the elements or configuration of FIG. 7.

“Oxygen saturation” refers to the amount of oxygen that is dissolved in arterial blood (written SpO2, SO2 or SaO₂). It is a measure of the percentage of hemoglobin binding sites in the bloodstream that, at the time of the observation, are transporting oxygen. A normal oxygen saturation range in human blood is around 94-98% at sea level, 92-94% at 5,000 ft., and <92% at higher elevations. Supplementary oxygen is highly desirable if blood oxygen saturation falls below 90% to counter the effects of hypoxemia.

“Functional oxygen saturation” is the ratio of oxyhemoglobin (HbO₂) to the sum of HbO₂+RHb (hemoglobin that is not carrying oxygen, called reduced hemoglobin). If the functional oxygen saturation is from arterial hemoglobin then it is called functional arterial oxygen saturation. Functional SO₂ is given by:

$$Functional {S O}_{2} = \frac{Hb O 2}{RHb + Hb O 2} \times 100. \qquad ( 1 )$$

This can be expressed in terms of concentrations as follows:

$$Functional {S O}_{2} = \frac{{C}_{{HbO}_{2}}}{{C}_{( RHb + {HbO}_{2} )}} \times 100 , \qquad ( 2 )$$

where C_{HbO}_{₂} is the concentration of HbO₂, and C_{(RHb+HbO}_{₂}₎ is the total concentration of RHb and HbO₂ in the blood.

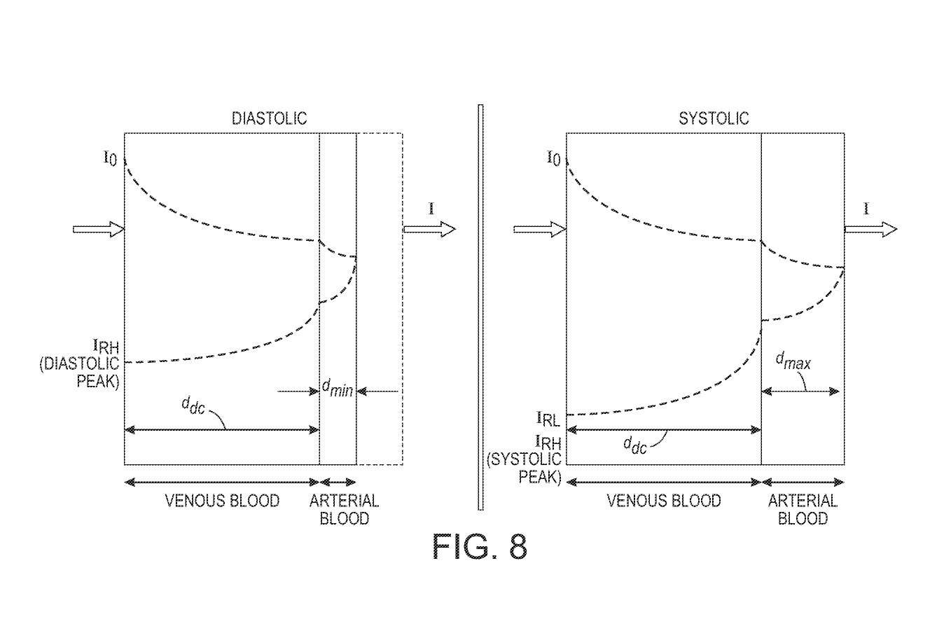

According to Beer's Law (also known as Beer-Lambert-Bouguer's law), as light travels through a media, the intensity I decreases exponentially with distance. FIG. 8 illustrates Beer's Law in a tissue layer containing venous and arterial structures which reflect light. If ε(λ) is the wavelength dependent extinction coefficient or absorptivity coefficient then:

I=I₀e^{-e(λ)cd} (5)

where I₀ is the detected intensity of incident light, c is the concentration in mmol/L (or g/dL), and d is the optical path length in cm.

The transmittance T is the ratio of transmitted light I to the amount of detected incident light I₀.

$$T = \frac{I}{{I}_{0}} = {e}^{- ɛ ( λ ) c d} \qquad ( 6 )$$

The absorbance A is given by:

A=−ln(T)=ε(λ)cd=α(λ)d (7)

where α(λ) is a measure of the rate of decrease in the intensity of light as it passes through a substance.

If multiple materials that absorb light at a given wavelength are present in the sample, the total absorbance Aₜ at wavelength λ is the sum of all absorbers:

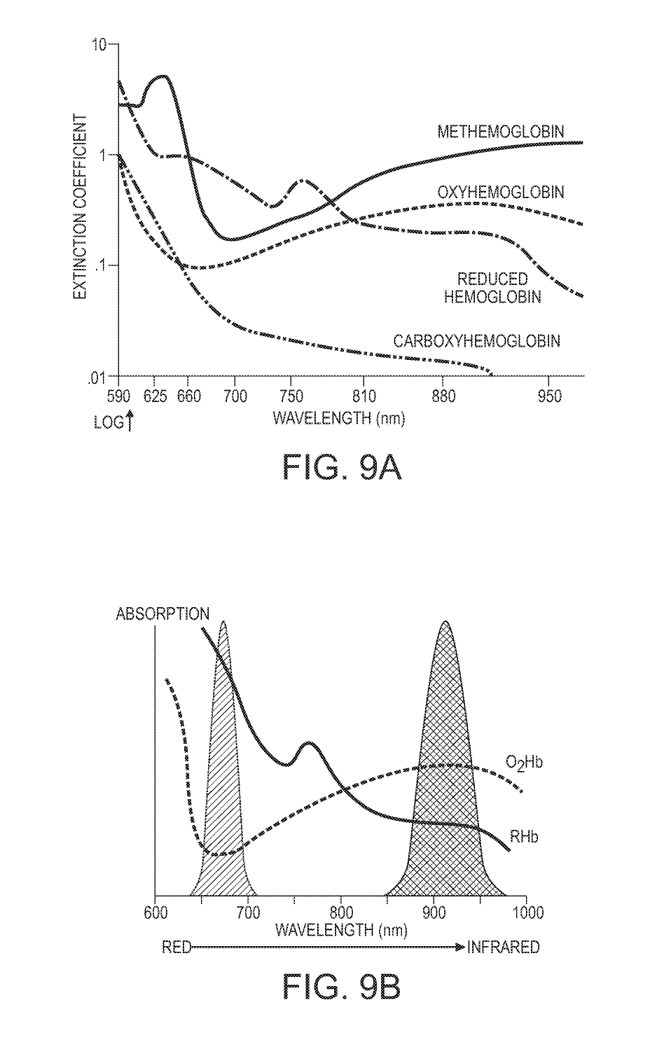

Aₜ=ε₁(λ)c₁d₁+ε₂(λ)c₂d₂+ε₃(λ)c₃d₃+ε₄(λ)c₄d₄ (6)

where extinction coefficients ε₁, ε₂, ε₃, ε₄ are respectively for RHb, HbO₂, COHb, and MetHb components selected at wavelength λ and are obtained using the extinction plot of FIG. 9A. Concentrations c₁, c₂, c₃, c₄ correspond to RHb, HbO₂, COHb, and MetHb, i.e., c₁=c_{RHb}, c₂=C_{HbO}_{₂}, c₃=C_{COHb}, and c₄=c_{MetHb}. The alternating part of absorbance allows pulsatile and non-pulsatile components to be differentiated.

During diastole, the reflected light from the arterial walls is retransmitted. There is absorbance again due to arterial and venous blood. Using Beer's law, considering multiple absorbers, peak intensities during diastole and systole can be written as follows. Light reflected from the living tissue (normally about 1 to 2% each way) is ignored. Peak intensity, I_{RH}, during diastole, (i.e., the minimum reflected light intensity) is given by:

$${I}_{RH} = {I}_{0} {e}^{- ( {ɛ}_{dc} ( λ ) {c}_{dc} 2 {d}_{dc} + ( {ɛ}_{Hb} ( λ ) {c}_{Hb} + {ε}_{{HbO}_{2}} ( λ ) {c}_{{HbO}_{2}} ) 2 {d}_{min} )} \qquad ( 7 )$$

where dₘᵢₙ is the diameter of arteries when it is minimal (i.e., diastole) where the absorbance due to arterial hemoglobin is minimal and corresponding intensity is at a maximum intensity I_{H}. It is to be noted that for transmission arrangement, effective transmission of light path will be once through the venous blood and once through the arterial blood. Hence the scalar value “2” will be replaced by “1” in Eqn. (7).

Peak intensity, I_{RL}, during systole, (i.e., the reflected light intensity in the return path), is given by:

$${I}_{RL} = {I}_{0} {e}^{- ( {ɛ}_{dc} ( λ ) {c}_{dc} 2 {d}_{dc} + ( {ɛ}_{Hb} ( λ ) {c}_{Hb} + {ε}_{{HbO}_{2}} ( λ ) {c}_{{HbO}_{2}} ) 2 {d}_{max} )} \qquad ( 8 )$$

where dₘₐₓ is the diameter of arteries when it is maximum and the corresponding light intensity is at a minimum intensity I_{L}. It is to be noted that for transmission arrangement, effective transmission of light path will be once through the venous blood and once through the arterial blood. Hence the scalar value “2” will be replaced by “1” in Eqn. (8).

Substituting dₘₐₓ=dₘᵢₙ+δd in Eqn. (8), where δd is the change in diameter of arteries during one cardiac cycle, and using Eqn. (7), we get:

$${I}_{RL} = {I}_{0} {e}^{- ( {ɛ}_{d c} ( λ ) {c}_{d c} 2 {d}_{d c} + ( {ɛ}_{Hb} ( λ ) {c}_{Hb} + {ε}_{{HbO}_{2}} ( λ ) {c}_{{HbO}_{2}} ) 2 {d}_{m i n} )} {e}^{- ( {ɛ}_{Hb} ( λ ) {c}_{Hb} + {ɛ}_{{HbO}_{2}} ( λ ) {c}_{{HbO}_{2}} ) 2 δ d} \qquad ( 9 )$$

Dividing Eqn. (6) by Eqn. (7) we get, the normalized transmitted signal due to variations in path length, Tₙ, where:

$${T}_{n} = \frac{{I}_{RL}}{{I}_{RH}} = {e}^{- ( {ɛ}_{Hb} ( λ ) {c}_{Hb} + {ɛ}_{{HbO}_{2}} ( λ ) {c}_{{HbO}_{2}} ) 2 δ d} \qquad ( 10 )$$

By the same argument as before, scalar value “2” will be replaced by “1” in Eqn. (10) for transmission arrangement. Ratio of systolic and diastolic peaks in Eqn. (10) removes the dependence on light intensities from illuminators at different wavelengths, if two or more LEDs are used. Eqn. (7) represents changes to reflected light caused by the pulsation of the blood in the arteries. The normalized absorption can be obtained using Eqn. (3) as follows:

$$\begin{matrix}{A}_{t} = - ln ( {T}_{n} ) \\ = - ln ( \frac{{I}_{RL}}{{I}_{RH}} ) \\ = ( {ɛ}_{Hb} ( λ ) {c}_{Hb} + {ɛ}_{{HbO}_{2}} ( λ ) {c}_{{HbO}_{2}} ) 2 δ d\end{matrix} \qquad ( 11 )$$

By the same argument as before, scalar value “2” will be replaced by “1” in Eqn. (11) for transmission arrangement. If we use two wavelengths, (i.e., red at 660 nm and IR at 940 nm), (FIG. 9B) and assume that the optical path lengths are the same, (i.e., both light passes through same size veins, arteries or arterioles), then the ratio of the absorbance at the red and IR wavelengths depends on the absorbers present in those components. Thus, we can write the ratio R as follows:

$$R = \frac{{A}_{t , Red}}{{A}_{t , IR}} = \frac{ln ( {I}_{RL , red} / {I}_{RH , red} )}{ln ( {I}_{RL , IR} / {I}_{RH , IR} )} \qquad ( 12 )$$

Substituting Eqn. (11) in Eqn. (12), we get a ratio:

$$R = \frac{{ɛ}_{Hb} ( {λ}_{Red} ) {c}_{Hb} + {ɛ}_{{HbO}_{2}} ( {λ}_{Red} ) {c}_{{HbO}_{2}} )}{{ɛ}_{Hb} ( {λ}_{IR} ) {c}_{Hb} + {ɛ}_{{HbO}_{2}} ( {λ}_{IR} ) {c}_{{HbO}_{2}} )} \qquad ( 13 )$$

Concentrations for hemoglobin and oxygenated hemoglobin can be expressed in terms of SO₂ using a revised version of Eqn. (2) in Eqn. (13). From Eqn. (2), we get the concentrations of HbO₂ and Hb as follows:

c_{HbO}^{₂}=SO₂(c_{HbO}^{₂}+c_{Hb}) (14A)

c_{Hb}=(1−SO₂)(c_{HbO}^{₂}+c_{Hb}) (14B)

Substituting Eqns. (14A & 14B) into Eqn. (13), we get a ratio:

$$R = \frac{( \begin{matrix}{ɛ}_{Hb} ( {λ}_{Red} ) ( 1 - {S O}_{2} ) ( {c}_{{HbO}_{2}} + {c}_{Hb} ) + \\ {ɛ}_{{HbO}_{2}} ( {λ}_{Red} ) {S O}_{2} ( {c}_{{HbO}_{2}} + {c}_{Hb} )\end{matrix} )}{( \begin{matrix}{ɛ}_{Hb} ( {λ}_{IR} ) ( 1 - {S O}_{2} ) ( {c}_{{HbO}_{2}} + {c}_{Hb} ) + \\ {ɛ}_{{HbO}_{2}} ( {λ}_{IR} ) {S O}_{2} ( {c}_{{HbO}_{2}} + {c}_{Hb} )\end{matrix} )} \qquad ( 15 )$$

Further simplification leads to cancellation of terms associated with hemoglobin concentrations. Rearranging terms, we can obtain the equation for the arterial oxygen saturation in percent:

$${S O}_{2} = \frac{[ {ɛ}_{Hb} ( {λ}_{Red} ) - R {ɛ}_{Hb} ( {λ}_{IR} ) ] \times 100}{( {ɛ}_{Hb} ( {λ}_{Red} ) - {ɛ}_{{HbO}_{2}} ( {λ}_{Red} ) ) + ( {ɛ}_{{HbO}_{2}} ( {λ}_{IR} ) - {ɛ}_{Hb} ( {λ}_{IR} ) ) R} \qquad ( 16 )$$

Eqn. (16) can also be written in simplified form as follows:

$${S}_{a} {O}_{2} = \frac{{k}_{2} - {k}_{4} R}{( {k}_{2} - {k}_{1} ) + ( {k}_{3} - {k}_{4} ) R} \times 100 = \frac{{k}_{2} - {k}_{4} R}{{\overline{k}}_{1} - {\overline{k}}_{3} R} \times 100 \qquad ( 17 )$$

It is to be noted that we use SO2 and SaO2 mean the same. In Eqn. (17), extinction coefficients are replaced by constants determined from clinical studies to produce a best fit between measured arterial functional oxygen saturation SₐO₂ using, for example, a FDA approved sensing method or in-vitro measurement of SₐO₂, and the ratio R of Eqn. (16).

### Example Flow Diagram

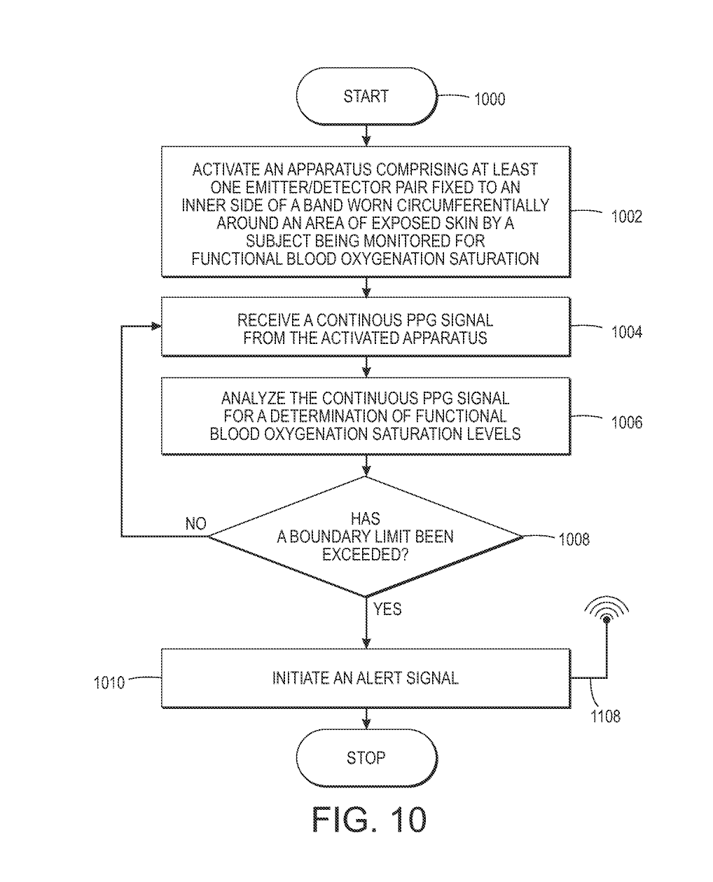

Reference is now being made to the flow diagram of FIG. 10 which illustrates one embodiment of the present method for monitoring a subject of interest for functional blood oxygen saturation. Flow processing begins at step 1000 and immediately proceeds to step 1002.

At step 1002, activate an apparatus comprising at least one emitter/detector pair fixed to an inner side of a band worn circumferentially around an area of exposed skin by a subject being monitored for functional blood oxygen saturation. The apparatus may be worn in any of the areas shown in FIGS. 1-3. The apparatus may be any of the transmissive or reflective devices of FIGS. 4 and 5. The device can be activated by the connection of power thereto or by pressing an ON/OFF switch such as Button 707 of FIG. 7. Upon activation, the illuminators emit their source light which, in turn, is sensed by each emitters paired photodetector. A continuous PPG signal is generated thereby.

At step 1004, receive a continuous PPG signal from the detectors of the activated apparatus.

At step 1006, analyze the continuous PPG signal for a determination of functional blood oxygen saturation level(s). Embodiments for determining a functional blood oxygen saturation level are discussed herein in detail.

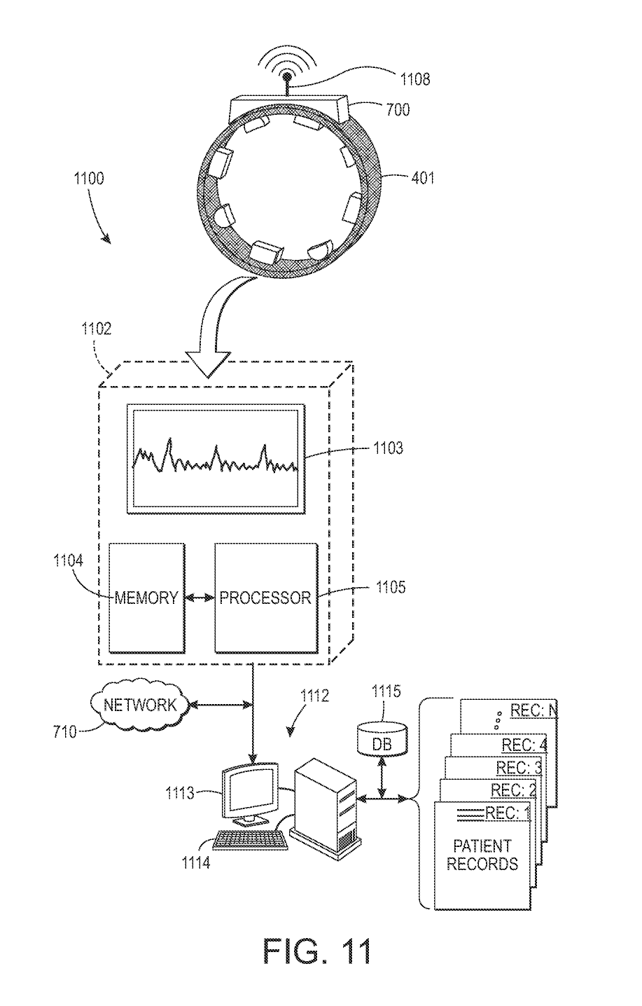

At step 1008, a determination is made, as a result of having determined the functional blood oxygenation level in step 1006, whether a boundary limit has been exceeded. If so then, at step 1010, an alert signal is initiated. The alert signal or notification can be sent to a technician, nurse, medical practitioner, and the like, using, for example, antenna 1108 (of FIG. 11). In one embodiment, the alert signal is communicated via network 710 of FIG. 7. Such a signal may take the form of a message or, for instance, a bell tone, ring, or sonic alert being activated at a nurse's station. The alert signal may take the form of initiating a visible light which provides an indication such as, for instance, a blinking colored light such as the LEDs 706 of FIG. 7. If, at step 1008, a boundary limit has not been exceeded then processing repeats with respect to step 1004 wherein the PPG signal is continuously received and analyzed for functional blood oxygen saturation. Processing repeats in a similar manner. In another embodiment, further processing stops. The apparatus hereof is intended to be used for continuous monitoring while the device is ON.

The flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Example Networked System

Reference is now being made to FIG. 11 which illustrates a block diagram of one example signal processing system 1100 for performing various aspects of the teachings hereof.

In FIG. 11, the control panel 700 of the present apparatus fixed to band 401 utilizes antenna 1108 to communicate a continuous PPG signal to a wireless cellular device 1102 which may be a smartphone, i-phone, Android Device, or another wireless cellphone as are widely used and commonly found in various streams of commerce. Smartphone 1102 has a display 1103, a memory 1104, and a processor 1105 which executes machine readable program instructions for analyzing the continuous PPG signal or for processing the time-series signal received from the present apparatus to extract the continuous PPG signal. The smartphone may execute applications developed and configured to work with various embodiments of the present transmissive or reflective sensing devices. Downloadable applications for a cellular smartphone 1102 may include, for example, a Signal Extractor Application which receives the time-series signal and extracts a physiological signal corresponding to one or more physiological functions which the subject is being monitored for. Another application may be a Signal Compensation Application which processes the PPG signal to compensate for artifacts that may have been introduced therein. A Signal Analyzer Application may be employed to analyze the PPG signal to determine the occurrence of a cardiac event for the subject. An Event Monitoring Application may be used for continuously determining whether the PPG signals are within acceptable limits. Such an application would initiate an alert in response to a physiological event having occurred or for being outside a boundary of acceptability pre-set for the subject. The Event Monitoring Application may be configured to communicate a message to a remote device such as the cellphone of a medical professional via antenna 1108. Such applications may be downloadable from an online AppStore where cellphone applications are often made available.

The networked system of FIG. 11 is shown in communication with a workstation 1112 comprising a computer case housing a motherboard, CPU, memory, interface, storage device, and a communications link such as a network card, and having a display device 1113 such as a CRT, LCD, or touchscreen display. An alphanumeric keyboard 1114 and a mouse (not shown) effectuate a user input. It should be appreciated that the workstation has an operating system and other specialized software configured to display a variety of numeric values, text, scroll bars, pull-down menus with user selectable options, and the like, for entering, selecting, or modifying information. The workstation has a removable media (not shown) and implements a database 1115 wherein various patient records are stored. Information obtained using the present apparatus can be uploaded to patient records. Records stored in the database can be indexed, searched, and retrieved in response to a query. Patient information can be stored to any of the records in the database and used for A-fib event monitoring. Although the database is shown as an external device, the database may be internal to the workstation mounted on a hard disk housed in the computer case. The processor 1105 and memory 1104 are in communication with the workstation via pathways (not shown) and may further be in communication with one or more remote devices over network 710. It should be appreciated that some or all of the functionality performed by the smartphone device 1102 may be performed, in whole or in part, by the workstation. Various aspects of the teachings hereof may be practiced in distributed environments where tasks are performed by a plurality of devices linked via a network and may be implemented using any known or later developed systems, structures, devices, or software by those skilled in the applicable arts without undue experimentation from the description provided herein.

One or more aspects of the systems and methods described herein are intended to be incorporated in an article of manufacture which may be shipped, sold, leased, or otherwise provided separately either alone or as part of a product suite. The above-disclosed features and functions or alternatives thereof, may be combined into other systems or applications. Presently unforeseen or unanticipated alternatives, modifications, variations, or improvements may become apparent and/or subsequently made by those skilled in the art and, further, may be desirably combined into other different systems or applications. Changes to the above-described embodiments may be made without departing from the spirit and scope of the invention. The teachings of any printed publications including patents and patent applications, are each separately hereby incorporated by reference in their entirety.

What is claimed is:

1. A method for monitoring a subject of interest for functional blood oxygen saturation, the method comprising:
activating an apparatus comprising at least one emitter/detector pair with at least two illuminators each emitting source light at a different wavelength band, and fixed to an inner side of a band worn circumferentially around an area of exposed skin by a subject of interest being monitored for functional blood oxygen saturation, each detector comprising at least one sensor that is sensitive to a wavelength band of a respective illuminator, each detector measuring an intensity of received light emitted by a respective illuminator, said measurements comprising a continuous photoplethysmographic (PPG) signal for said subject, each illuminator being separated from its paired detector by a distance D; and
analyzing said continuous PPG signal for a determination of functional blood oxygen saturation.

2. The method of claim 1, wherein said apparatus is a transmissive device where distance D separating each illuminator and paired detector defines a chord of living tissue through which said emitted source light passes, said distance being less than 75% of a diametrical distance of an area where said band is being worn, each detector measuring an intensity of light passing through said chord of living tissue.

3. The method of claim 1, wherein said apparatus is a reflective device, distance D is a distance between each illuminator and paired detector as measured around said circumference, said emitted light impacting said skin surface at an angle θ_{L}, each detector measuring an intensity of light reflecting off a surface of skin at an angle θ_{R}, where 0°<(θ_{L}, θ_{R})<90°.

4. The method of claim 1, wherein multiple emitter/detector pairs are fixed circumferentially around an inner side of said band.

5. The method of claim 1, wherein a wavelength band of a first illuminator is centered about 660 nm and a wavelength band of a second illuminator is centered about 940 nm.

6. The method of claim 1, wherein each emitter/detector pair is configured to emit/detect a different wavelength band.

7. The method of claim 1, wherein said functional blood oxygen saturation comprises:
$$Functional {S O}_{2} = \frac{{C}_{{HbO}_{2}}}{{C}_{( RHb + {HbO}_{2}} )} \times 100$$
where C_{HbO}_{₂} is a total concentration of HbO₂, and C_{(RHb+HbO}_{₂}₎ is a sum of the concentrations of RHb and HbO₂.

8. The method of claim 1, further comprising:
processing signals received by said detectors to generate a ratio; and
calibrating said ratio to an approved standard.

9. The method of claim 4, further comprising:
obtaining information from each of said emitter/detector pairs; and
increasing an accuracy of said measurements by performing any of: averaging signals, discarding signals, weighting signals based on a statistical analysis, and discarding intensity values determined to be below a level of acceptability.

10. The method of claim 1, further comprising any of:
activating said illuminators to emit source light for a desired length of time; and
turning said illuminators ON/OFF according to a pre-defined interval.

11. The method of claim 1, further comprising communicating said functional blood oxygen saturation to a remote device comprising any of: a smartphone, a Wi-Fi router, an I-Pad, a Tablet-PC, a laptop, a remote server, and a desktop computer.

12. The method of claim 11, said remote device further comprising any of: a USB connection, memory, a transmitter, a receiver, a display, a storage device, and a connection for delivering power from said remote device.

13. The method of claim 11, wherein said remote device turns said illuminators ON/OFF according to a pre-defined schedule.

14. The method of claim 11, wherein said communication occurs in response to said functional blood oxygen saturation being outside a pre-defined limit of acceptability.

15. The method of claim 11, wherein said communication comprises any of: text, email, picture, graph, chart, and pre-recorded message.

16. An apparatus for monitoring a subject of interest for functional blood oxygen saturation, the apparatus comprising:
at least one emitter/detector pair with at least two illuminators each emitting source light at a different wavelength band, and fixed to an inner side of a band worn circumferentially around an area of exposed skin by a subject of interest being monitored for functional blood oxygen saturation, each detector comprising at least one sensor that is sensitive to a wavelength band of a respective illuminator, each detector measuring an intensity of received light emitted by a respective illuminator, said measurements comprising a continuous photoplethysmographic (PPG) signal for said subject, each illuminator being separated from its paired detector by a distance D; and
a processor executing machine readable program instruction for analyzing said continuous PPG signal for a determination of functional blood oxygen saturation.

17. The apparatus of claim 16, wherein said apparatus is a transmissive device where distance D separating each illuminator and paired detector defines a chord of living tissue through which said emitted source light passes, said distance being less than 75% of a diametrical distance of an area where said band is being worn, each detector measuring an intensity of light passing through said chord of living tissue.

18. The apparatus of claim 16, wherein said apparatus is a reflective device, distance D is a distance between each illuminator and paired detector as measured around said circumference, said emitted light impacting said skin surface at an angle θ_{L}, each detector measuring an intensity of light reflecting off a surface of skin at an angle θ_{R}, where 0°<(θ_{L}, θ_{R})<90°.

19. The apparatus of claim 16, wherein multiple emitter/detector pairs are fixed circumferentially around an inner side of said band.

20. The apparatus of claim 16, wherein a wavelength band of a first illuminator is centered about 660 nm and a wavelength band of a second illuminator is centered about 940 nm.

21. The apparatus of claim 16, wherein each emitter/detector pair is configured to emit/detect a different wavelength band.

22. The apparatus of claim 16, wherein said functional blood oxygen saturation comprises:
$$Functional {S O}_{2} = \frac{{C}_{{HbO}_{2}}}{{C}_{( RHb + {HbO}_{2} )}} \times 100$$
where C_{HbO}_{₂} is a total concentration of HbO₂, and C_{(RHb+HbO}_{₂}₎ is a sum of the concentrations of RHb and HbO₂.

23. The apparatus of claim 16, said processor further performing:
processing signals received by said detectors to generate a ratio; and
calibrating said ratio to an approved standard.

24. The apparatus of claim 19, said processor further performing:
obtaining information from each of said emitter/detector pairs; and
increasing an accuracy of said measurements by performing any of: averaging signals, discarding signals, weighting signals based on a statistical analysis, and discarding intensity values determined to be below a level of acceptability.

25. The apparatus of claim 16, said processor further performing any of:
activating said illuminators to emit source light for a desired length of time; and
turning said illuminators ON/OFF according to a pre-defined interval.

26. The apparatus of claim 16, further comprising communicating said functional blood oxygen saturation to a remote device comprising any of: a smartphone, a Wi-Fi router, an I-Pad, a Tablet-PC, a laptop, a remote server, and a desktop computer.

27. The apparatus of claim 26, said remote device further comprising any of: a USB connection, memory, a transmitter, a receiver, a display, a storage device, and a connection for delivering power from said remote device.

28. The apparatus of claim 26, wherein said remote device turns said illuminators ON/OFF according to a pre-defined schedule.

29. The apparatus of claim 26, wherein said communication occurs in response to said functional blood oxygen saturation being outside a pre-defined limit of acceptability.

30. The apparatus of claim 26, wherein said communication comprises any of: text, email, picture, graph, chart, and pre-recorded message.
